(19) United States
(12) Patent Application Publication
(10) Pub. No.: US 2020/0222077 A1
(43) Pub. Date: Jul. 16, 2020

(54) PREMIER CATHETER ACCESS PUNCTURE TOOL, SYSTEM AND METHODS

(71) Applicant: Vtail Medical LLC, Lake Forest, CA (US)

(72) Inventor: Randall Takahashi, Lake Forest, CA (US)

(21) Appl. No.: 16/740,424

(22) Filed: Jan. 11, 2020

Related U.S. Application Data
(60) Provisional application No. 62/791,081, filed on Jan. 11, 2019.

(51) Int. Cl. A61B 17/34; A61B 17/3209

(52) U.S. Cl. CPC A61B 17/3415; A61B 2017/00778; A61B 17/3496; A61B 17/32093

(57) ABSTRACT

Premier Catheter Access Puncture Tool includes a device mounted retractable blade assembly which travels over-the-wire in conjunction with a procedure wherein precise and controlled cutting on the skin facilitates better access to the site of ingress with minimal soft tissue injury or insult. Smart versions are likewise disclosed.

### BACKGROUND OF THE DISCLOSURE

The present invention relates to access devices for interventional procedures using catheters. Specifically, the instant disclosure concerns any catheter based procedures whereby access to lumens of the human body is facilitated by inserting catheters, introducers, guide-wires, trocars and any related medical devices into such vessels, such ingress comprising access through or under skin, dermis or any related tissues, organs and systems of bodies of living, frozen or suspended animals.

### OBJECTS AND SUMMARY OF THE INVENTION

Briefly stated, Premier Catheter Access Puncture Tool comprising at least a device mounted retractable blade assembly, with a spring-means and a first and second position, for being disposed over-the-wire (OTW) and cutting skin to house other medical devices at an access site.

According to embodiments, there is provided premier catheter access puncture tool, which comprises, in combination, a device mounted retractable blade assembly, a spring-means for releasing the blade assembly from a first, or retracted position to a second, or extended position and, a track-based positioning system effective to be inserted over-the-wire (OTW), being an assembly for housing the retractable blade allowing it to have a low profile.

According to embodiments, there is provided a system for enlarging catheter access sites traveling over-the-wire and spring-means actuated.

According to embodiments there is provided a tissue dilator with skin cutter which is tapered and has a wire lumen, the exterior has skin cutting blade(s) which may be retracted and are practical for arterial access sites particularly.

According to embodiments at least a device mounted retractable blade assembly having a spring-means. From at least a first to a second position to make a precise incision increasing bore-area and space for micro-catheter ingress and the like access needs;

According to embodiments, any appropriate access site, for example, femoral, radial, brachial, or other peripheral vascular wherein catheter-mounted, driven and/or over-the-wire types of systems are giving ingress to body lumen of animals.

### BRIEF DESCRIPTION OF THE FIGURES/DRAWINGS

Various preferred embodiments are described herein with references to the drawings in which merely illustrative views are offered for consideration, whereby:

FIG. 1 comprises a stick figure showing radial, brachial, femoral and/or other peripheral vascular access sites; and

FIG. 2 is a schematic showing embodiments of devices, systems and methods of the present inventions, as disclosed and claimed herein;

FIG. 3 is a schematic showing embodiments of the prior art to devices, systems and methods of the present inventions, as disclosed and claimed herein;

FIG. 4 is a schematic showing embodiments of a device, systems and methods of the present inventions, as disclosed and claimed herein;

FIG. 5 is a schematic showing embodiments of the device, systems and methods of the present inventions, as disclosed and claimed herein;

FIG. 6 is a schematic showing embodiments of the device, systems and methods of the present inventions, as disclosed and claimed herein;

FIG. 7 is a schematic showing embodiments of the device, systems and methods of the present inventions, as disclosed and claimed herein;

FIG. 8 is a schematic showing embodiments of the device, systems and methods of the present inventions, as disclosed and claimed herein;

FIG. 9 is a schematic showing embodiments of the device, systems and methods of the present inventions, as disclosed and claimed herein;

FIG. 10 is a schematic showing embodiments of the device, systems and methods of the present inventions, as disclosed and claimed herein;

FIG. 11 is a schematic showing embodiments of the device, systems and methods of the present inventions, as disclosed and claimed herein;

FIG. 12 is a schematic showing embodiments of the device, systems and methods of the present inventions, as disclosed and claimed herein;

FIG. 13 is a schematic showing embodiments of the device, systems and methods of the present inventions, as disclosed and claimed herein.

Corresponding reference characters indicate corresponding components throughout the several views of the drawings. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention.

Also, common but well-understood elements that are useful or necessary in a commercially feasible embodiment are often not depicted in order to facilitate a less obstructed view of these various embodiments of the present invention.

### DETAILED DESCRIPTION OF THE DISCLOSURES

The present inventors have discovered a way to minimize trauma, damage insult and injury to tissue at vascular access sites in the human body or any other system in need of the same. The present invention differs materially from the prior art in that it is expressly designed for arterial puncture wherein there is no needle movement once a target site is acquired to avoid losing said target. Several of the below patents are different for want of this desideratum, otherwise having had identified aspects of the problem solved by the instant invention, it is respectfully proposed.

Expressly incorporated herein by reference are the following US Letters Patents, each as if fully set forth herein, albeit reviewed and materially different each from aspects of the present invention: U.S. Pat. Nos. 9,743,953; 9,743,952; 9,480,497; 9,468,405; 8,845,671; 8,753,271; 8,663,263; 8,628,469; 8,562,521; 8,512,363 and 5,843,108.

The present inventors have discovered that premier catheter puncture systems enable improved access and/or ingress to catheter driven interventional and/or surgical sites by enabling both accurate and precise placement of blade-means within a spacing-means loaded type of sheath that fits over-the-wire such that enlargement of an access site is seamless and does not damage skin any more than needed.

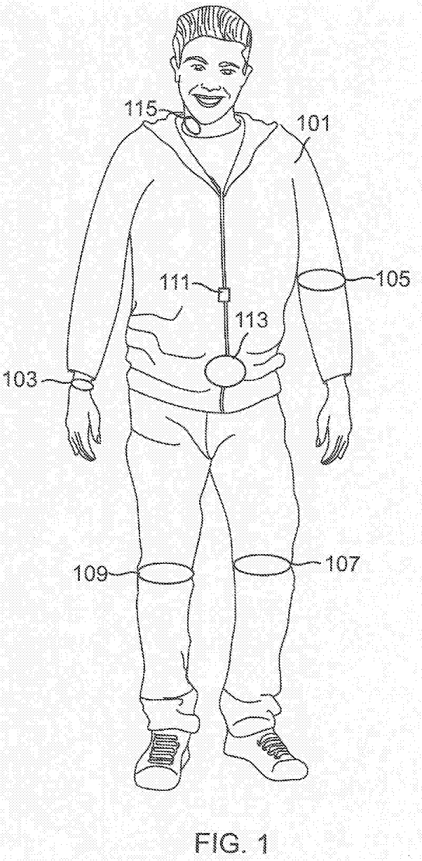

For example, turning now to FIG. 1, a cartoon-version of a patient (prior to being prepared for a procedure) 101 is shown with various possible access points shown from radial 103 to brachial 105, femoral 107, carotid 115, other peripheral vascular 109 or related sites such as adjacent to the spinal column 111 or renal 113. Those skilled in the art understand any site or ingress site may be appropriate, depending upon procedural and patient specific criteria. Arterial access at these sites in what the present invention is ideally suited for as discussed above and claimed below.

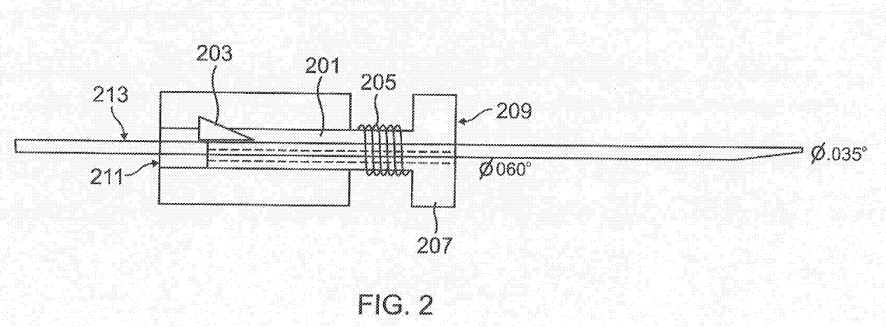

Referring now also to FIG. 2, an exploded and completely schematic version is shown in a second position for illustration only. In the view blade 203 is positional within housing 201 whereby being positioned over guide-wire 213 maintains a coaxial orientation such that spring-means 205 or any suitable substitute allows actuation, or movement of blade 203 from at least a first-retracted position (not shown) to a second or extended position, relative to proximal and 211 and distal and 209 of assembly at housing 201. Those skilled in the art likewise understand distal and plug 207 at distal and 209 may move to release blade 203 whereby it penetrates a dermal layer, or skin beneath which it is disposed on guide-wire 213 near proximal and of housing 201 via spring-means 205 located at distal and 209. Exemplary but not limiting values for the ranges and tolerances, such as used with proto-typed versions show actual reduction to practice and completely reliable spring action is proven, (Validated & Improved by M4D, Lake Forest, Calif. 92630).

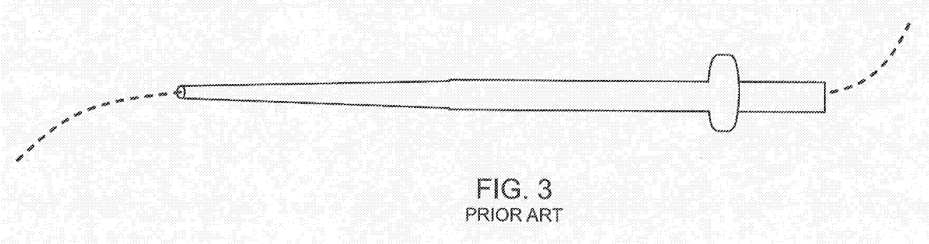

Referring now also to FIGS. 3 and 4-9, a cartooned Prior Art device which is replaced by the instant teachings is shown as a schematic of a conventional tissue dilator used for vessel puncture and sheath insertion. As artisans understand pitfalls and shortcomings of the same the guidewire (99 from here on it, lumen for a wire 100, stopper 102 and handle grip 104) require no further description.

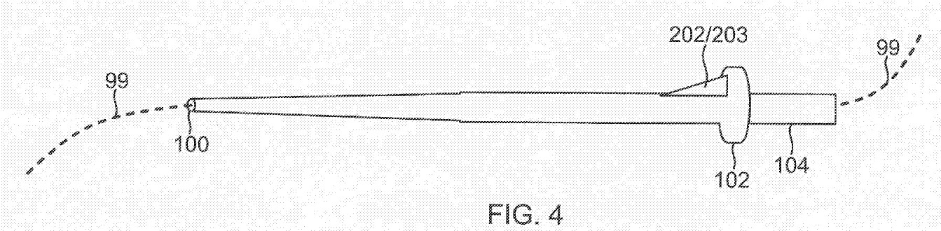

FIG. 4 shows an inventive tissue dilator with puncture point skin cutter first, disposed about guide wire 99, wherein wire lumen 100 extends through the device. Skin cutting blade(s) 202/203 project coaxially from stopper 102 and handle grip 104. A skilled operator realizes that this allows blade(s) 202/203 to be aligned and make incisions accurately with improved precision relative to the target access site. Sliceage means cutting with a minimal collateral damage according to the instant teachings where a minimally optimized incision is made.

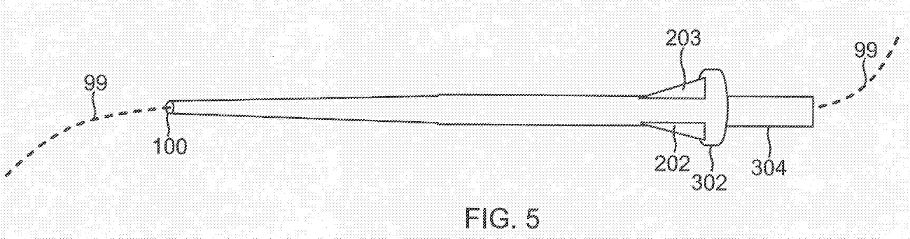

FIG. 5 likewise shows an inventive device which is a tissue dilator with puncture point skin cutter second (two blades). The system is disposed about guide wire 99, wherein wire lumen 100 extends through the device. Skin cutting blade(s) 202/203 project coaxially from stopper 302 and handle grip 304. A skilled operator realizes that this allows blade(s) 202/203 to be aligned and make incisions accurately with improved precision relative to the target access site.

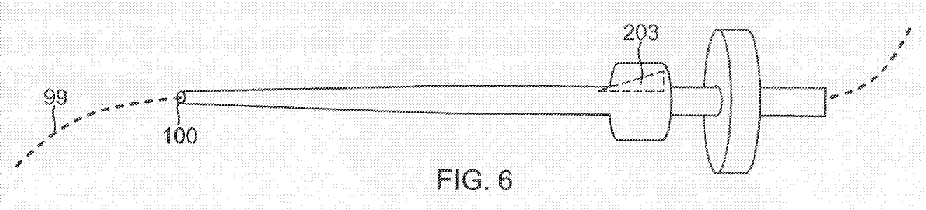

FIG. 6 likewise shows an inventive device which is a tissue dilator with puncture point skin cutter. The system is disposed about guide wire 99, wherein wire lumen 100 extends through the device. Skin cutting blade 203 is shown in a first position where it is retracted before being extended coaxially from stopper 302 and handle grip 304. A skilled operator realizes that this allows blade(s) 202/203 to be aligned and make incisions accurately with improved precision relative to the target access site.

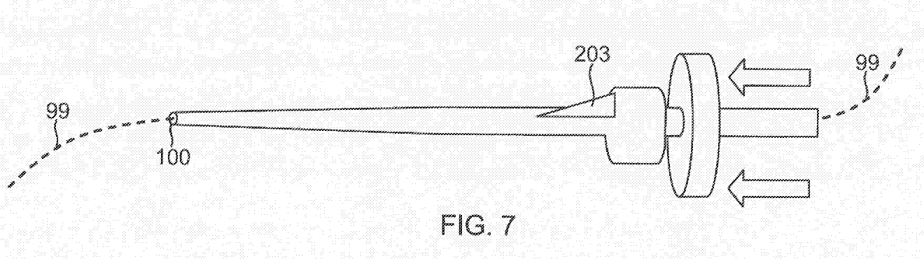

FIG. 7 likewise shows an inventive device which is a tissue dilator with puncture point skin cutter. The system is disposed about guide wire 99, wherein wire lumen 100 extends through the device. Skin cutting blade 203 is pushed out into a second position where it projects coaxially based upon the force shown moving it from the first (FIG. 6) to second (FIG. 7) position. A skilled operator realizes that this allows blade(s) 202/203 to be aligned and make incisions accurately with improved precision relative to the target access site.

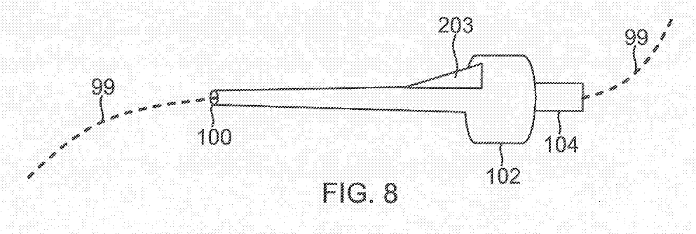

FIG. 8 likewise shows an inventive device which is a tissue dilator with puncture point skin cutter second. The system is disposed about guide wire 99, wherein wire lumen 100 extends through the device. Skin cutting blade(s) 202′203 project coaxially from stopper 102 and handle grip 104. A skilled operator realizes that this allows blade(s) 202/203 to be aligned and make incisions accurately with improved precision relative to the target access site.

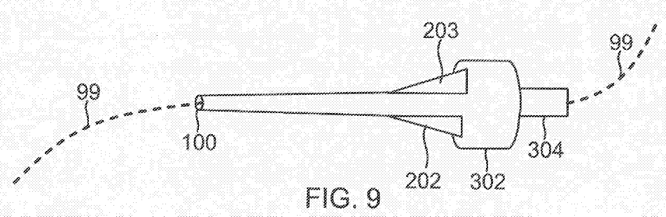

FIG. 9 likewise shows an inventive device which is a tissue dilator with puncture point skin cutter second (two blades). The system is disposed about guide wire 99, wherein wire lumen 100 extends through the device. Skin cutting blade(s) 202/203 in the second position shown project coaxially from stopper 302 and handle grip 304. A skilled operator realizes that this allows blade(s) 202/203 to be aligned and make incisions accurately with improved precision relative to the target access site.

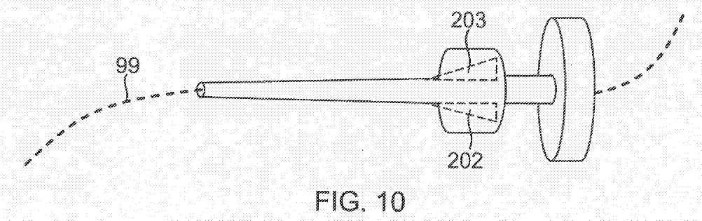

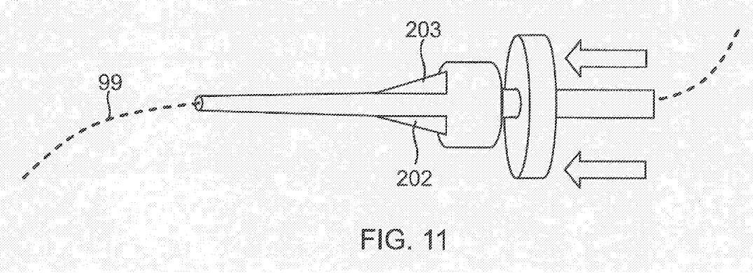

FIG. 10 likewise shows an inventive device which is a tissue dilator with puncture point skin cutter third FIG. 5 likewise shows an inventive device which is a tissue dilator with puncture point skin cutter second (retractable multiple blades design). The system is disposed about guide wire 99, wherein wire lumen 100 extends through the device. Skin cutting blade(s) 202/203 are retracted in a first position as opposed to FIG. 11 where they project coaxially from stopper 302 and handle grip 304. A skilled operator realizes that this allows blade(s) 202/203 to be aligned and make incisions accurately with improved precision relative to the target access site.

FIG. 11 likewise shows an inventive device which is a tissue dilator with puncture point skin cutter third (retractable multiple blades design). The system is disposed about guide wire 99, wherein wire lumen 100 extends through the device. Skin cutting blade(s) 202/203 are pushed out namely thy project coaxially from stopper 302 and handle grip 304, in a second position. A skilled operator realizes that this allows blade(s) 202/203 to be aligned and make incisions accurately with improved precision relative to the target access site.

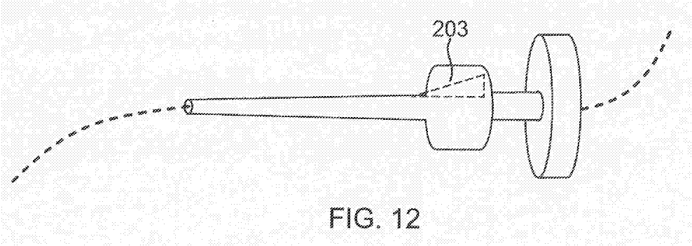

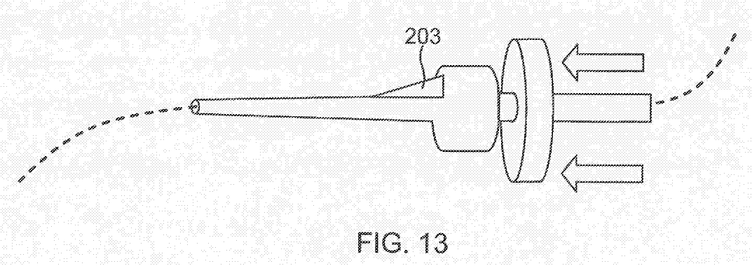

FIG. 12 likewise shows an inventive device which is a tissue dilator with puncture point skin cutter fourth. FIG. 12 likewise shows an inventive device which is a tissue dilator with puncture point skin cutter fourth (retractable single blade design). The system is disposed about guide wire 99, wherein wire lumen 100 extends through the device. Skin cutting blade(s) 202/203 are retracted in a first position as opposed to FIG. 13, in a second position where they project coaxially from stopper 302 and handle grip 304. A skilled operator realizes that this allows blade(s) 202/203 to be aligned and make incisions accurately with improved precision relative to the target access site.

Those skilled in the art readily understand that the instant system, devices and methods work for any interventional, minimally invasive surgical procedure or the like.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An I/O mechanism may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antenna, and a network interface device (e.g., a network interface card (NIC), Wi-Fi card, cellular modem, data jack, Ethernet port, modem jack, HDMI port, mini-HDMI port, USB port), touchscreen (e.g., CRT, LCD, LED, AMOLED, Super AMOLED), pointing device, trackpad, light (e.g, LED), light/image projection device, or a combination thereof.

Memory according to the invention refers to a non-transitory memory which is provided by one or more tangible devices which preferably include one or more machine-readable medium on which is stored one or more sets of instructions (e.g., software) embodying any one or more of the methodologies or functions described herein. The software may also reside, completely or at least partially, within the main memory, processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof. In certain embodiments, a device of the invention includes a tangible, non-transitory computer readable medium for memory. Exemplary devices for use as memory include semiconductor memory devices, (e.g., EPROM, EEPROM, solid state drive (SSD), and flash memory devices e.g., SD, micro SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A system for enhancing access for a procedure, comprising, in combination:
at least a blade-means housed within an assembly, which assembly travels over-the-wire (OTW) in conjunction with the use of catheters to cut skin, during an access procedure, in a coaxial fashion with the orientation of the wire, for use within a patient, whereby the system is tapered and has a wire lumen and exterior blade(s) which may be moved from a first to a second position.

2. The system of claim 1, for any procedure selected from the group of interventional, minimally inclusive, surgical, or other invasive and transdermal procedure using at least one of radial, femoral, brachial, peripheral vasculature-related and renal access points.

3. The system of claim 1, said blade-means housed within an assembly being at least a spring-loaded blade.

4. The system of claim 3, further comprising the spring-loaded blade having a triangular orientation in a second or extended position.

5. Premier catheter access puncture tool, comprising, in combination:
A device mounted retractable blade assembly;
A spring-means for releasing the blade assembly from a first, or retracted position to a second, or extended position;
A track-based positioning system effective to be inserted over-the-wire (OTW), being an assembly for housing a retractable blade allowing it to have a low profile.

6. The device of claim 5, and systems of claims 1-4 and the figures and appendix, whereby low-profile means working dimensions such as a 0.060 catheter tapering to 0.035 as known to artisans.

7. The system of claim 6, whereby minimal tissue insult or injury occurs at the access site because the movement or travel of the blade is limited by the mechanical nature of the blade assembly relative to a guide wire, the coaxial alignment and movement from a first to a second position.

8. The system of claim 7, disclosed herein disposed is a modular radial plug housing effective for OTW radial access.

9. An improved arterial puncture access tool comprising, in combination:
A recessed blade housed within a blade assembly which is a low profile plug, which is OTW allowing for minimal skin damage when the access site has to be expanded owing to precise and accurate slicing of the skin proximal to the site of catheter or other device ingress.

10. The improved arterial puncture access tool, of claim 9, further comprising:
The recessed blade moves from a first retracted, to a second extended position while maintained in tight orientation relative to the puncture target.

11. The improved arterial puncture access tool, of claim 10, further comprising:
More than one recessed blade, which moves from a first retracted, to a second extended position while maintained in tight orientation relative to the puncture target.

12. Improved methods for enhancing vascular arterial access comprising, in combination:
providing a catheter access puncture tool for radial, femoral and related access needs whereby said tool is coaxial with a guide-wire allowing for precise and controlled sliceage of dermis above below and around access apertures and ports without making larger punctures than needed to allow passage of required devices therein.

13. The method of claim 12, whereby the catheter access puncture tool further comprises a spring-loaded retractable single blade, moving from a first to a second position along the guide wire.

14. The method of claim 12, whereby the catheter access puncture tool further comprises a spring-loaded retractable double blade, moving from a first to a second position along the guide wire.

15. The method of claim 13, whereby the catheter access puncture tool further comprises a single blade having a fixed position.

16. The method of claim 14, whereby the catheter access puncture tool further comprises a double blade having a fixed position.

17. The method of claim 13, whereby the catheter access puncture tool further comprises a spring-loaded retractable single blade, moving from a first to a second position along the guide wire, the coaxial nature of the alignment and control of the blade able to expand the aperture with a ‘skin nick’ precisely placed without concomitant tissue insult or injury.

18. The method of claim 14, whereby the catheter access puncture tool further comprises a spring-loaded retractable double blade, moving from a first to a second position along the guide wire, the coaxial nature of the alignment and control of the blade able to expand the aperture with a ‘skin nick’ precisely placed without concomitant tissue insult or injury.

19. The method of claims 17 and 18, further comprising triangular blades.

20. Methods as disclosed in claim 19, coupled with smart cutting and imaging tools and on board processors to harvest, manage and learn from data related to each access attempt and resultory data set, by storing said data and using it for subsequent procedures and/or the patients personalized data sets.
